(19) United States
(12) Patent Application Publication
(10) Pub. No.: US 2019/0183562 A1
(43) Pub. Date: Jun. 20, 2019

(54) SKIN WRINKLE TREATMENT

(71) Applicant: LIFE CARE MEDICAL DEVICES LIMITED, New Smyrna Beach, FL (US)

(72) Inventor: Alan David WIDGEROW, Irvine, CA (US)

(21) Appl. No.: 16/219,092

(22) Filed: Dec. 13, 2018

Related U.S. Application Data
(60) Provisional application No. 62/599,089, filed on Dec. 15, 2017.

(51) Int. Cl. A61B 18/14; A61B 18/20; A61B 18/12

(52) U.S. Cl. CPC A61B 18/14; A61B 2018/0047; A61B 18/1206; A61B 18/203

(57) ABSTRACT

A method for treating skin may first involve contacting an applicator of a skin heating device with an epidermal layer of the skin. The applicator may be activated to generate high-frequency alternating magnetic fields. The applicator may be used to heat a dermal layer of the skin, with the high-frequency alternating magnetic fields, to a temperature of less than 40 degrees Celsius, to stimulate a release of heat shock protein in the dermal layer to promote collagen generation. The method may also involve limiting heating of the dermal layer of the skin to less than 40 degrees Celsius.

### CROSS-REFERENCE TO RELATED APPLICATIONS

This application claims the benefit of U.S. Provisional Patent Application No. 62/599,089, filed Dec. 15, 2017, entitled, “SKIN WRINKLE TREATMENT.” The disclosure of this priority application is hereby incorporated by reference in its entirety into the present application.

### FIELD

This application is related to medical methods and systems. Specifically, the application relates to a method and system for treating skin to reduce the appearance of wrinkles.

### BACKGROUND

Skin wrinkles are often the consequence of advancing age and sun exposure. With increasing age and excessive sun exposure, skin quality deteriorates. This is due, in part, to changes in hydration and epidermal thickness, and on a molecular scale, to a decrease in the amount of collagen in the dermis. Further, subcutaneous fat accumulates or atrophies, leading to furrowing of the skin, which produces wrinkles. In today's society, the appearance of skin wrinkles is often viewed negatively, and there is a desire in the community for a means to safely reduce or eliminate wrinkles.

Many different treatments have been used or attempted for reducing skin wrinkles or the appearance of skin wrinkles for cosmetic purposes. Many different cosmetics have been tried, for example, such Retin-A™ (tretinoin) and other vitamin A derivatives. Injected substances, such as BOTOX™ (botulinum toxin), have also been used to reduce skin wrinkles. Additionally, various medical devices and procedures have been used to reduce wrinkles, such as chemical peels, dermabrasion, laser skin resurfacing and the like. More recently, ultrasound has been used, in an attempt to provide specific and localized treatment to the dermis. Though specificity is improved, ultrasound may cause cavitation of fat cells with lipid leakage and cell destruction. The process may be associated with significant pain and unpredictable fat loss.

In general, the methods developed thus far for skin wrinkle removal or reduction are either ineffective, invasive or both. Cosmetic products like such Retin-A™ often have little or no effect. Injected substances like as BOTOX™ (botulinum toxin) must be repeated every few months and also sometimes have minimal effect. More invasive procedures, such as laser resurfacing, chemical peels and dermabrasion, require significant recovery and may even permanently scar the epidermis and/or the dermis.

Therefore, it would be advantageous to have devices and methods for improved skin wrinkle treatment. Ideally, these devices and methods would be minimally invasive and would also be effective.

### BRIEF SUMMARY

In one aspect of the present disclosure, a method for treating skin may involve: contacting an applicator of a skin heating device with an epidermal layer of the skin; activating the applicator to generate high-frequency alternating magnetic fields; heating a dermal layer of the skin, with the high-frequency alternating magnetic fields, to a temperature of less than 40 degrees Celsius, to stimulate a release of heat shock protein in the dermal layer to promote collagen generation; and limiting heating of the dermal layer of the skin to less than 40 degrees Celsius. In some embodiments, the method may involve moving the applicator multiple times over a treatment area of the skin during one skin treatment, so that the treatment area is contacted and heated multiple times during the one skin treatment. The method may also be repeated at least one time at a later date.

In some embodiments, activating the applicator involves delivering a frequency of 25-30 megahertz. In some embodiments, activating the applicator involves delivering a thermal power of 150-200 Watts. The method may also optionally include sensing the temperature of the dermal layer with a temperature sensor. In some embodiments, the method may also include automatically deactivating the applicator when the dermal layer reaches a temperature of at least 40 degrees Celsius. In some embodiments, the method may include automatically reducing an energy level of the applicator when the dermal layer reaches a temperature of at least 40 degrees Celsius. In some embodiments, a temperature sensor is attached to the applicator. The method may further include sensing a temperature of the epidermal layer of the skin and extrapolating it to the dermal temperature.

In another aspect of the present disclosure, a skin treatment system may include an applicator configured to generate high-frequency alternating magnetic fields. The applicator may include a temperature sensor. The system also includes a controller coupled to the activator, and the controller includes a processor containing computer-executable programming instructions for providing a skin treatment via the applicator. The instructions are executable by the processor for: activating the applicator to generate the high-frequency alternating magnetic fields; heating the dermal layer of the skin, with the high-frequency alternating magnetic fields, to a temperature of less than 40 degrees Celsius, to stimulate a release of heat shock protein in the dermal layer to promote collagen generation; and limiting heating of the dermal layer of the skin to less than 40 degrees Celsius.

In some embodiments, the processor is further configured to receive a sensed temperature of the dermal layer from the temperature sensor, and the step of limiting heating of the dermal layer is based at least in part on the received sensed temperature. In one embodiment, the temperature sensor is a thermocouple attached to the applicator. In some embodiments, the computer-executable programming instructions are further configured to automatically deactivate the applicator when the dermal layer reaches a temperature of at least 40 degrees Celsius. In some embodiments, the computer-executable programming instructions are further configured to automatically reduce an energy level of the applicator when the dermal layer reaches a temperature of at least 40 degrees Celsius. In some embodiments, the temperature sensor is further configured to sense a temperature of the epidermal layer of the skin. The controller may activate the applicator to deliver a frequency of 25-30 megahertz and a thermal power of 150-200 Watts.

These and other aspects and embodiments are described in further detail below, in reference to the attached drawing figures.

### BRIEF DESCRIPTION OF THE DRAWINGS

FIG. 1 is a perspective view of a skin treatment system, according to one embodiment;

FIG. 2 is a flow diagram, illustrating a skin treatment method, according to one embodiment;

FIG. 3 is a cross-sectional view of a skin treatment applicator, according to one embodiment;

FIGS. 4A and 4B are rear and side views, respectively, of a skin treatment applicator, according to an alternative embodiment;

FIG. 5 is a cross-sectional view of a distal end of a skin treatment applicator, according to one embodiment; and

FIG. 6 is a rear, partially cross-sectional view of a skin treatment applicator, according to one embodiment.

### DETAILED DESCRIPTION

The present disclosure describes methods and devices for treatment of tissues in a subject/patient, preferably for cosmetic treatment of skin, with a high degree of specificity for the dermis. The skin treatment system includes a magnetic induction applicator with a geometry to provide a concentrated and intense alternating magnetic field to shallow layers of skin, when placed in close proximity to the skin. The method involves creating a high-frequency alternating magnetic field that, when directed in proximity with tissue, results in the production of heat through inductive coupling with the tissue, thus resulting in the desired biologic effect. Representative examples of such biologic effects include, but are not limited to, release of heat shock proteins, tissue contraction or shrinkage, and induction of a wound response that leads to other biomolecular changes. Generally, application of the high frequency alternating magnetic field itself may induce, or the concomitant production of heat may induce, the movement of a charged species or other biomolecule or bioactive molecule or species within the tissue leading to various biological responses, such as, but not limited to, the production of collagen by cells and dermal thickening.

The described skin treatment method and system may be used to improve the cosmetic appearance of the skin by controllably heating a superficial layer of skin, preferably the dermis. The system may be used for the direct heating of moist conductive tissues, such as the viable dermis, during cosmetic skin treatment. The system generally heats tissues of low conductivity, such as the epidermis, less efficiently than it heats the dermis, thus providing a safer skin treatment. The method and system minimize the risk of significant burns to the skin surface, and eliminate charring and the generation of smoke, as they do not rely on capacitive coupling for their effects. The patient is isolated from the electrical current, and no electrical current is conducted from the applicator or the patient.

Generally, the devices provided herein include a means for generating and applying a high frequency alternating magnetic field, a means for controlling the alteration of the tissue or biomolecule contained therein, a means for monitoring feedback related to heat generation, and a means for housing the device. Particularly, the devices may be handheld, such that a handheld piece incorporates a source of radiofrequency electrical energy coupled to a coil and an impedance matching network to produce an alternating magnetic field. When tissue is brought into proximity of the alternating magnetic field, heating of the tissue results as a consequence of either or both of dipole formation and oscillation or eddy current formation.

Optionally, the system and method may also provide for tissue cooling, to remove or disperse heat from the coil, the source of RF electrical energy, and/or the surface of the skin. For example, a disposable or permanent tip or cover placed between the induction coil and the skin may provide a thermally conductive surface that absorbs and distributes heat from the skin surface, similar to a heat sink. Alternatively, a thermally conductive substance may be placed on the skin or tissue. The system and method may also include feedback monitoring of heat generation, eddy current formation in the tissue, ultrasound detection of tissue alterations, changes in impedance in tissues that lead to an impedance mismatch between the magnetic field applicator and the radiofrequency generator, hydration, and/or the like, to provide for adjustable control of inductively generated heat in the tissue and/or device.

The skin treatment method and system described herein provide a number of potential advantages, such as but not limited to being non-invasive, not requiring electrical contact with the body/skin of the subject, and providing controllable heating only to a thin layer of tissue. The method and system may be useful not only for cosmetic procedures, such as facial rejuvenation and wrinkle treatment, but potentially for other beneficial effects caused by induction of biomolecular events, such as a wound response, production of heat-shock proteins or an inflammatory response in tissue.

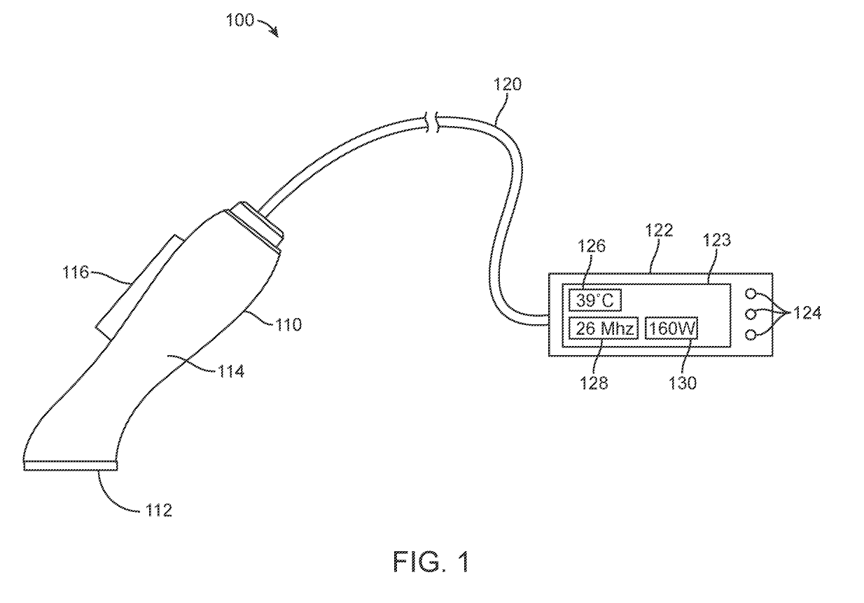

Referring to FIG. 1, in one embodiment, a skin treatment system 100 includes an applicator 110 and a combined radiofrequency (RF) generator and controller 122 (herein referred to either as “RF generator” or “controller”), which are connected to one another via a cable 120. Applicator 110, which will be described in further detail below, includes a handle 114 (or “housing”), a skin contact surface 112, and a coolant reservoir 116. Controller 122 includes a display 123 and multiple control knobs 124 (or switches, buttons or the like). Display 123 may include any suitable combination of data, such as skin temperature 126, frequency 128 and power 130. Some embodiments may include an RF generator and a separate controller (in other words, two separate devices). Alternative embodiments may use and deliver a different form of energy to the skin or other treatment tissue, such as but not limited to radiant energy, plasma energy, acoustic energy, or bipolar or monopolar electrosurgical energy.

One exemplary embodiment of applicator 110 is described in detail in U.S. Patent Application Pub. No. 2011/0077451, filed Nov. 23, 2010, titled “Inductive Heating of Tissues Using Alternating Magnetic Fields and Uses Thereof,” which is hereby fully incorporated by reference into this application.

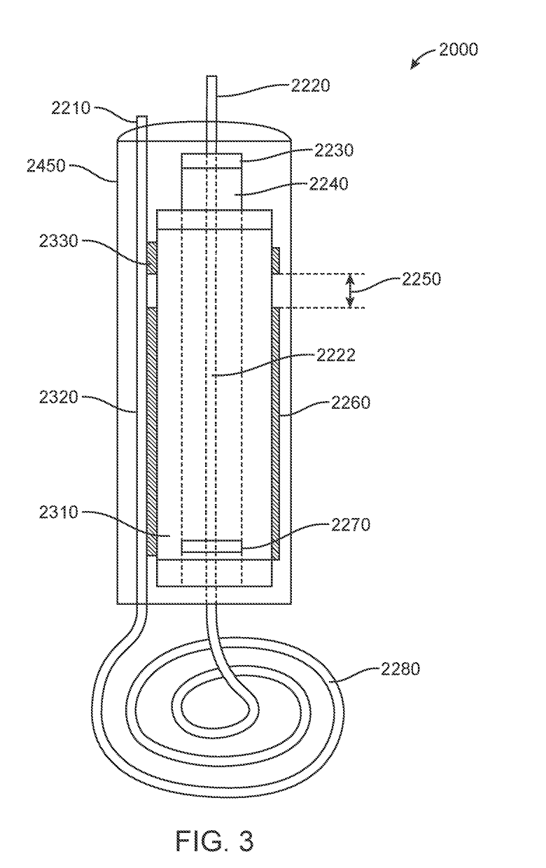

FIG. 3 illustrates one application of an applicator 2000 in further detail, per U.S. Patent Application Pub. No. 2011/0077451. FIG. 3 is a cross-sectional view of applicator 2000 used to produce a magnetic field. A center copper tube 2220, which is configured to conduct RF electricity and coolant fluid (or “refrigerant”), is surrounded by a polytetrafluoroethylene (PTFE) cylinder 2240. Endcaps 2230 and 2270 position and hold copper tube 2220 within PTFE cylinder 2240. Copper tube 2220 includes a first straight portion 2222, a coil 2280 at the distal end of applicator 2000, and a second straight portion 2320 (or “return portion”), which runs in parallel with first straight portion 2222 and ends in an exit port 2210 at the proximal end of applicator 2000. The coolant is directed in through first straight portion 2222, through coil 2280, and out through second straight portion 2320 and exit port 2210.

In order to provide impedance matching between an RF generator 122 and applicator 2000, a ceramic insulator 2310 is positioned around PTFE cylinder 2240. Ceramic insulator 2310 has two capacitor rings 2330 and 2260 made of copper pipe. Second straight portion 2320 is in electrical contact with capacitor rings 2330 and 2260. By adjusting a spacing 2250 between capacitor rings 2330 and 2260, the impedance match between RF generator 122 and applicator 2000 can be affected. Applicator 2000 is encased in a copper cylinder 2450 (or “housing”), which is attached to the ground shield of the coaxial wire (not shown), in order to shield any stray radiated RF.

As mentioned above, controller 122 may include a source of RF energy. Examples of RF generators include those sold by Comdel, Inc. (Gloucester, Mass.). The electrical output of controller/generator 122 is coupled to applicator 2000, which may further be part of an impedance matching network that may additionally include a capacitor. The source of energy used may be a constant current or a constant voltage power supply or may be a modulated current or a modulated voltage power supply. Controller/generator 122 may be able to produce RF energy with a power in the range 10-10,000 W, and more preferably in the range of about 100 to about 1000 W, and even more preferably in the range of about 150 to about 200 W. The power-supply may typically operate at frequencies of about 100 kHz to about 5.8 GHz, and more preferably in the range of about 1 MHz to about 5.8 GHz, and even more preferably in the range of about 25 MHz to about 30 MHz. Beneficially, the RF generator may be frequency-agile, that is, as the impedance of the load changes, the frequency output of RF generator 122 changes slightly to provide a better impedance match between the load and generator 122 and thus maintain the output power within a controllable tolerance.

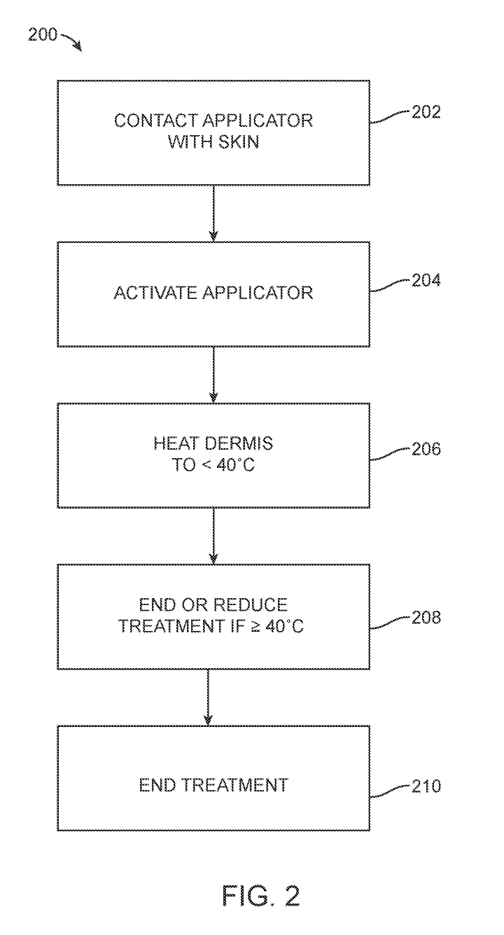

Referring now to FIG. 2, a skin treatment method 200, which may be performed using any suitable embodiment of skin treatment system 100, will now be described. In one embodiment, the first step 202 of method 200 is to contact skin treatment surface 112 of applicator 110 with the patient's skin. For example, applicator 110 may be used to treat the patient's face in some embodiments. The second step 204 may be to activate applicator 110, to begin delivering energy to the skin. In an alternative embodiment, the order of the first step 202 and the second step 204 may be switched, so that applicator 110 is activated before it is contacted with the skin. In either case, in the third step 206, applicator 110 may be held in contact with the skin until the dermis is heated to a temperature equal to or less than 40 degrees Celsius. Heating the dermis to this temperature and no greater than this temperature may be beneficial for skin wrinkle reduction or smoothing, because it appears to stimulate the release of heat shock protein in the dermis. In addition to being effective, heating the dermis to only 40 degrees Celsius or less typically will not stimulate nerve fibers in the dermis to sense pain, so the treatment is painless for most if not all people.

The fourth step 208 of the method 200 is to automatically stop or reduce the delivery of RF energy if the patient's dermis reaches a temperature of 40 degrees Celsius or of greater than 40 degrees Celsius, depending on the embodiment. In other words, in some embodiments the acceptable temperature threshold includes 40 degrees, while in other embodiments the acceptable temperature is less than 40 degrees. In either case, system 100 will automatically either shut off or reduce the power of energy delivery when the threshold is reached. Finally, the last step 210 is the end of treatment. In various embodiments, any given treatment area, such as an area of the face, may be heated using applicator 110 multiple times during one treatment. For example, applicator 110 may be moved over the face multiple times during one treatment. Additionally, treatments may be repeated at intervals. For example, it may be beneficial to have one treatment per week for a period of a month or several months to achieve a desired skin appearance.

Controller 122 may include a processor with computer-readable instructions in the form of software, which direct controller 122 to control a given treatment delivered by applicator 110. In some embodiments, for example, applicator 110 may include a heat sensor, such as but not limited to a thermocouple, for sensing a temperature of the epidermis and/or the dermis of the patient's skin. This sensed temperature is transmitted from applicator 110 to controller 122, and the processor of controller 122 will automatically shut off or turn down the energy delivered to applicator 110 when the sensed temperature reaches or exceeds 40 degrees Celsius. Controller 122 may also be programmed to deliver energy in a certain power range, such as about 150 to about 200 W, and in a certain frequency range, such as about 25 MHz to about 30 MHz. In some embodiments, controller 122 may also guide a treatment in other ways. For example, it may include a timer function, to suggest or require a given treatment time or to allow a physician to enter a desired treatment time. Controller 122 may also suggest a pattern in which to treat a given area of skin. In some embodiments, controller 122 may provide one or more alerts or alarms (audible, visual or both) when a certain temperature is reached, when a certain amount of time has elapsed, and/or the like.

As will be described further below, applicator 110 may include any of a number of different heat sensor devices. In some cases, applicator 110 may sense the temperature of the epidermis, and the processor of controller 122 may estimate or extrapolate an approximate temperature of the dermis from the sensed epidermal temperature. In any given alternative embodiment, the steps of the method 200 may be rearranged, one or more steps may be eliminated, or one or more additional steps may be added, without departing from the scope of this disclosure.

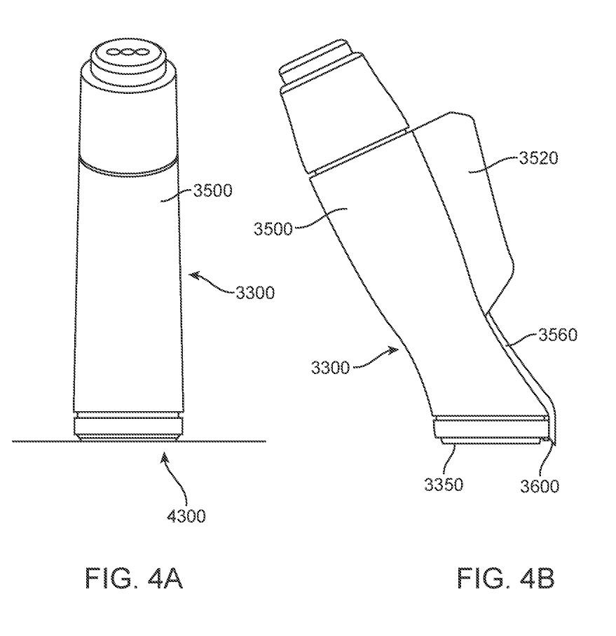

FIGS. 4A and 4B are back and side views, respectively, of an alternative embodiment of an applicator 3300. In this embodiment, the housing 3500 of applicator 3300 may be made of an electrically non-conductive material, such as plastic. Housing 3500 is attached to a shield 3350 (or “tip”) configured to contact the skin 4300. Shield 3350 may be disposable, to help maintain cleanliness of applicator 3300. Shield 3350 may also be thermally conductive, which may serve to disperse or distribute heat. Optionally, applicator 3300 may incorporate a coolant, for example R-134a, contained in a second coolant housing 3520 and directed through a solenoid and pipe 3560 to an exit nozzle 3600. This coolant can be controllably directed to the treated tissue before, during and/or after the treatment, in order to limit the heating of the very superficial skin.

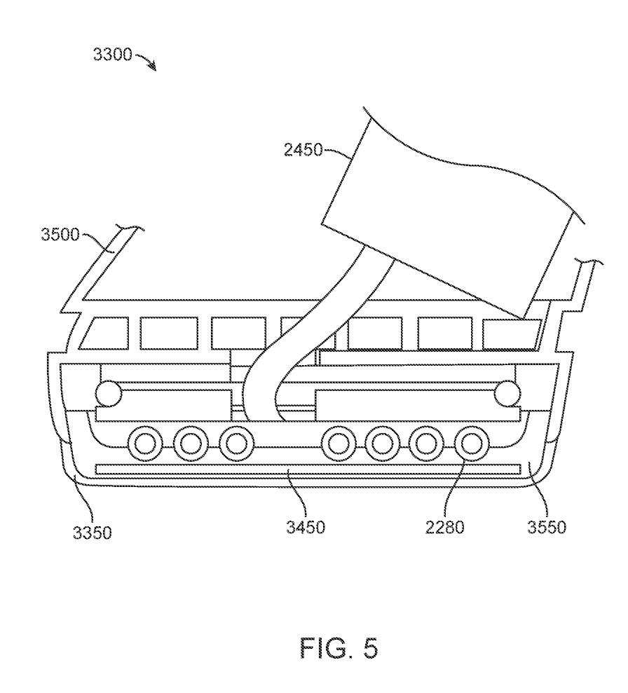

Referring now to FIG. 5, a cross-sectional view of an endplate 3550 of one embodiment of a distal end of applicator 3300 is shown. Endplate 3550 is in intimate thermal contact with the coil 2280, which has circulating refrigerant or coolant within it, to cool endplate 3550. This coolant may optionally be supplied from a circulating chiller using water and antifreeze. Alternatively, gas such as air, nitrogen, Freon™, R-12, R-134a, or carbon dioxide, could serve the purpose of cooling. An optional, removable shield or tip 3350 may be in intimate contact with the endplate 3550, thereby providing a means for the coolant to maintain an ambient temperature in the shield or tip 3350.

FIG. 5 also shows an optional Faraday shield 3450, which is a conductive element intended to reduce capacitive coupling of coil 2280 to the subject and thus minimize any stray electric field. Note that cooling endplate 3550 may be in intimate contact with coil 2280, in order to provide optimal thermal conduction. This may be accomplished by molding or by forming endplate 3550 to partially or completely surround coil 2280.

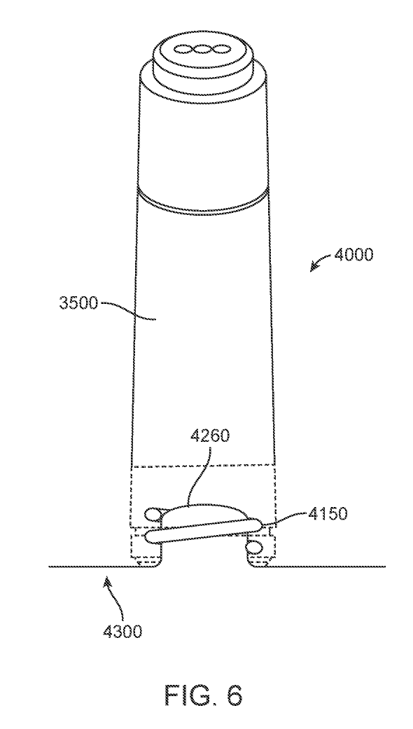

FIG. 6 shows a partial view of another alternative embodiment of an applicator 4000 incorporating a two-turn solenoid coil 4150, the bore of which surrounds a small mound of skin 4260 that protrudes up from the skin surface 4300 into the interior (or “bore”) of the solenoid coil 4150, as a result of negative pneumatic pressure generated within the housing 3500. As the magnetic field within the turns of the solenoid coil 4150 is much stronger than the field outside of a pancake coil, and the geometry of the skin portion 4260 within the bore is different than the flat skin surface 4300, the power required to obtain an effect is reduced, and the effect on the skin portion 4260 can be more beneficial. Alternatively, a mechanical tissue-shaper may be positioned in contact with the skin 4300.

In various embodiments, energy may be inductively, conductively or radiatively applied, in combination with the use of an alternating magnetic field. Additionally, the energy may be pulsed in order to improve the thermal kinetics of the tissue heating. Examples of types of energy that might be applied, according to various embodiments, include but are not limited to RF energy, radiant energy and vibrational energy. The RF energy may have a frequency from about 20 kHz to about 40 GHz, and may be applied using coils, electrodes or one or more antennae. The radiant energy may have a wavelength from about 600 nm to 11 nm. The vibrational energy may be sonic or ultrasonic, with a frequency from about 20 Hz to 80 MHz. In the case of inductive heating, the energy may not be incident on the target, but may be induced in the target to be converted from one form to another.

Combined devices, using induction plus an energy source, e.g., laser or ultrasound, may enhance the effects or be used to combine certain treatments. Because the interaction of laser energy and ultrasound waves with tissue is physically different than the present invention, synergistic and combination effects are possible. For example, it may be desirable to treat the stratum corneum and epidermis with an alternate form of energy simultaneously, or sequentially, while treating with magnetic induction, thereby resulting in greater volumetric treatment of the skin. An applicator of radiant energy may include an optical assembly that focuses the radiant energy on the relevant target or below the target surface to get a sub-surface effect and spare the superior surface. A pressure-wave applicator may consist of a focused ultrasound transducer, which is coupled to the target tissue with an acoustic impedance matching material, such as gelatin, mineral oil or glycerin.

In some embodiments, the device includes a mechanism for monitoring the progress of the treatment effect in tissue. For example, the alternating magnetic field results in tissue heating, and this in turn alters the electrical properties of the target tissue. The tissue heating may have effects that alter electrical properties of the tissue either transiently or permanently. The effects may include, for example, dehydration, coagulation and/or rearrangement of molecules. These changes may be detected as a change in impedance or by monitoring hydration or eddy current formation. For example, as the impedance of the skin changes, the impedance match between the RF generator and applicator 110 is typically altered, and this change may be detected through changes in power absorption or frequency. This change can alternatively be used as a signal to determine the level or duration of application of energy. Alternatively, the change in tissue can be detected using ultrasound to detect morphological changes. Change in the flow of eddy currents may be detected as an indicator of the progress of reaction and may reflect morphological changes, dehydration and/or heat in the system.

As the tissue treatment process is initiated, coil 2280, endplate 3550, and skin may increase in temperature. When RF energy delivery to the skin ceases, the temperature will fall. Such temperature changes can be monitored by devices, such as thermocouples or thermistors. Sometimes, such devices may behave erratically in the presence of strong electromagnetic fields. Therefore, devices such as infrared thermometers may be more suitable to monitor the temperatures. These may be placed distal to the source of radiation, and the signals may be transmitted through fiber optics.

Another approach to monitoring heat at the site of treatment involves monitoring temperature sensitive chemicals placed at the treatment site. These heat sensitive chemicals may be in the form of liquid crystals disposed on a film and placed in contact with the treatment site. The liquid crystals may change color upon reaching certain temperature thresholds and may be monitored by collecting and detecting reflected light. Transducers may be used in the system to monitor the temperature and/or power output of the RF generator, the reflected power into the generator, the presence of water flow into the applicator and/or the generator. The presence of a short-circuit anywhere in the system may be indicated by a rapid rise in current in the generator and/or applicator.

Another example of an optional safety feature is a mechanical or electrical transducer between a disposable shield 3350 and housing 3500 of applicator 3300 (FIGS. 4A and 4B). In such embodiments, if shield 3350 is not present (connected to housing 3500), the RF generator will not engage/activate. Encoding of the interlock in shield 3350 ensures that a particular shield 3350 is only used on a particular patient. Multiple interlocks may incorporated into applicator 3300 to prevent running the device with shield 3350 removed. Some embodiments may include one or more thermal switches, which will power down/shut off the device if overheating occurs. Fast breakers quickly cut off the output if a power-output transient occurs. System 100 may also optionally include a foot pedal, to minimize the possibility of unintentional activation of the system 100.

In some embodiments, the induced magnetic field in applicator 3300 may be actuated or amplified upon detection of a load. A relatively small current may be applied to applicator 3300 when it is not in proximity to a tissue target. As applicator 3300 comes near or contacts the target, the change in impedance is detectable, and this may be used as a signal to increase power to applicator 3300. In this example, applicator 3300 in place on the skin is matched as a unit, i.e. skin and applicator 3300, to achieve the impedance match with the power supply. Where there is a mismatch, power will not be delivered. This safety feature minimizes the exposure of applicator 3300 components to significant power load when applicator 3300 is not applied to tissue, thus potentially reducing wear of the device, as well as protecting the patient and operator.

Skin wrinkle treatment system 100 may be used to induce changes in tissue (typically skin) by applying alternating magnetic fields to the tissue, such that currents are induced within the tissue (eddy currents). These currents encounter resistance, which results in the generation of heat. These eddy currents form most efficiently where there is a moist, polar environment, thus enabling electron displacement or ion flow within the target. Thus, for example, in skin, the generation of eddy currents in the moist underlying dermis is favored over the superficial dehydrated epidermis and stratum corneum layers, and the underlying, more non-polar adipose layer. This differential in conductivity results in preferable heating of conductive (dermis) tissues over less conductive tissues.

Sufficient induced energy is required to generate enough heat to overcome the effects of cooling from the body and blood flow. Therefore, coil 2280 is configured to place the magnetic field in close proximity to the skin surface and deliver a high frequency, intense alternating magnetic field, such that heating in the dermis is rapid and specific. Geometric considerations include the size and shape of coil 2280, which may be selected to minimize distance between coil 2280 and the skin target. Rapid and specific heating of the dermis, as is achieved using an intense alternating magnetic field, minimizes the total heat volume that collateral tissues are subject to.

Variations in cooling of the skin surface may be achieved by increasing the volume of coolant to applicator 3300 or to surface of the skin. These variations may be optimized to provide additional protection of the tissue proximal to applicator 3300 from the effects of heating.

Disposition of a heat sink between coil 2280 and tissue may provide dispersion and distribution of excess heat. As the treatment progresses, heat may build up inside the tissue and at the interface between the skin and applicator 3300, thereby heating shield 3350. The presence of a thermally conductive mass between the surface of the tissue and shield 3350 serves to minimize heating of applicator 3300, and may additionally serve to maintain an ambient temperature at the tissue surface.

In certain instances, coil geometry may result in uneven heating at the tip of applicator 3300. For example, eddy currents may form in the skin, which reflect the shape of coil 2280. Particularly where coil 2280 is shaped as a toroid, a ring-shaped heating pattern may result in the tissue. The removal of heat in this instance serves to minimize the build-up of excess heat, i.e. the heat is distributed more evenly at the skin surface.

A skin treatment method may involve inducing heat in the dermis of a subject to affect a biological response. The method may involve applying a high frequency alternating magnetic field to the skin to inductively heat the dermis, thereby affecting the biological response. In some embodiments, the method may involve mechanically or pneumatically shaping an area of skin prior to applying the high frequency alternating magnetic field. Some embodiments may further involve applying radiant energy, acoustic energy or vibrational energy to the skin concurrently or sequentially with the high frequency alternating magnetic field.

In some embodiments, the method may further involve monitoring feedback from the magnetic field and adjusting an amount of the heat induced in the dermis, based at least in part on the feedback. Feedback monitoring may involve, for example, detecting heat in the dermis, detecting eddy currents formed in the dermis, detecting hydration changes in the dermis, and/or detecting impedance changes in the dermis. In some embodiments, detecting heat in the dermis may involve monitoring heat sensitive liquid crystal media or monitoring infrared radiation. Feedback monitoring may further involve monitoring heat generated by another source of radiant energy, plasma energy, acoustic energy, and/or bipolar or monopolar electrosurgical energy.

In some embodiments, the method may involve cooling the dermis to disperse heat. For example, cooling the dermis may involve positioning a thermally conductive surface in contact with the skin and/or an exterior surface of an inductive coil generating the alternating magnetic field. A thermally conductive surface may be a heat sink that passively disperses heat, may have a circulating coolant, may be a cryogenic material or may have a cryogenic material disposed thereon.

The induced biological response may include, for example, activation of heat shock protein, induction of wound response, production of collagen, alteration of a biomolecule (or biomolecules) and/or the like. Inductive heating may also activate collagen repair and/or tissue growth, which may result in a smoothing of the skin or reduction in skin laxity.

The above description is believed to be a complete and accurate description of various aspects and embodiments of a skin treatment method and system. Description of the embodiments is not intended to limit the scope of the present invention as defined by the following claims.

We claim:

1. A method for treating skin, the method comprising:
contacting an applicator of a skin heating device with an epidermal layer of the skin;
activating the applicator to generate high-frequency alternating magnetic fields;
heating a dermal layer of the skin, with the high-frequency alternating magnetic fields, to a temperature of less than 40 degrees Celsius, to stimulate a release of heat shock protein in the dermal layer to promote collagen generation; and
limiting heating of the dermal layer of the skin to less than 40 degrees Celsius.

2. The method of claim 1, further comprising moving the applicator multiple times over a treatment area of the skin during one skin treatment, so that the treatment area is contacted and heated multiple times during the one skin treatment.

3. The method of claim 2, further comprising repeating the method at least one time at a later date.

4. The method of claim 1, wherein activating the applicator comprises delivering a frequency of 25-30 megahertz.

5. The method of claim 4, wherein activating the applicator comprises delivering a thermal power of 150-200 Watts.

6. The method of claim 1, further comprising sensing the temperature of the dermal layer with a temperature sensor.

7. The method of claim 6, further comprising automatically deactivating the applicator when the dermal layer reaches a temperature of at least 40 degrees Celsius.

8. The method of claim 6, further comprising automatically reducing an energy level of the applicator when the dermal layer reaches a temperature of at least 40 degrees Celsius.

9. The method of claim 6, wherein the temperature sensor is attached to the applicator.

10. The method of claim 6, further comprising:
sensing a temperature of the epidermal layer of the skin; and
estimating the temperature of the dermal layer based on the sensed temperature of the epidermal layer.

11. A skin treatment system, comprising:
an applicator configured to generate high-frequency alternating magnetic fields, wherein the applicator includes a temperature sensor;
a controller coupled to the activator, wherein the controller includes a processor containing computer-executable programming instructions for providing a skin treatment via the applicator, the instructions being executable by the processor for:
activating the applicator to generate the high-frequency alternating magnetic fields;
heating a dermal layer of the skin, with the high-frequency alternating magnetic fields, to a temperature of less than 40 degrees Celsius, to stimulate a release of heat shock protein in the dermal layer to promote collagen generation; and
limiting heating of the dermal layer of the skin to less than 40 degrees Celsius.

12. The system of claim 11, wherein the processor is further configured to receive a sensed temperature of the dermal layer from the temperature sensor, and wherein the step of limiting heating of the dermal layer is based at least in part on the received sensed temperature.

13. The system of claim 12, wherein the temperature sensor is a thermocouple attached to the applicator.

14. The system of claim 12, wherein the computer-executable programming instructions are further configured to automatically deactivate the applicator when the dermal layer reaches a temperature of at least 40 degrees Celsius.

15. The system of claim 12, wherein the computer-executable programming instructions are further configured to automatically reduce an energy level of the applicator when the dermal layer reaches a temperature of at least 40 degrees Celsius.

16. The method of claim 12, wherein the temperature sensor is further configured to sense a temperature of the epidermal layer of the skin, and wherein the processor is configured to estimate a temperature of the dermal layer, based on the sensed temperature of the epidermal layer.

17. The system of claim 11, wherein the controller activates the applicator to deliver a frequency of 25-30 megahertz.

18. The system of claim 17, wherein the controller activates the applicator to deliver a thermal power of 150-200 Watts.
